(19) United States
(12) Patent Application Publication
(10) Pub. No.: US 2022/0240766 A1
(43) Pub. Date: Aug. 4, 2022

(54) Endoscope Kit having Functions of Injection, Clamping and Placing Medical Materials or Medicines

(71) Applicants: Chin-Piao Chang, Miaoli County (TW); Hong-So Kao, Taipei City (TW)

(72) Inventors: Chin-Piao Chang, Miaoli County (TW); Hong-So Kao, Taipei City (TW)

(21) Appl. No.: 17/165,964

(22) Filed: Feb. 3, 2021

(51) Int. Cl. A61B 1/018; A61B 17/34; A61B 1/00; A61B 1/06; A61B 18/24; A61B 10/06; A61B 10/04; A61B 18/14; A61M 31/00

(52) U.S. Cl. CPC A61B 1/018; A61B 17/3478; A61B 1/00016; A61B 1/0684; A61B 18/24; A61B 1/00045; A61B 2018/00589; A61B 10/04; A61B 18/1492; A61B 1/00032; A61B 1/00128; A61M 31/00; A61B 1/00096; A61B 10/06

(57) ABSTRACT

The present invention provides an endoscope kit comprising a working instrument, an endoscope and a Y joint. The working instrument comprises an operation part, a rod portion and a handle, and the operation part and the handle respectively connected to the two ends of the rod portion. The endoscope comprises a cannula, a light source input end and an output. The Y joint comprises a first branch, a second branch and a third branch; the first branch has two interfaces for connecting the working instrument and the injection needle to the endoscope; and the second branch has an injection interface. The light source input end is used to input a light source and transmit the light source to the cannula. Therefore, the endoscope kit of the present application can be used to process biological tissues after the working instrument is installed in the endoscope, and the injection port is used to inject medicines for treatment.

### (a) TECHNICAL FIELD OF THE INVENTION

The present invention relates to an endoscope kit having functions of injection, clamping, and placing medical materials or medicines, and more particularly to an endoscope combined with a working instrument. After the endoscope is positioned, the working instrument of the endoscope kit can be directly used to process biological tissues.

### (b) DESCRIPTION OF THE PRIOR ART

With the continuous advancement of surgical methods, traditional open surgery has advanced to the current minimally invasive surgery, for example, arthroscopic surgery and laparoscopic surgery have widely adopted minimally invasive surgery methods. In minimally invasive surgery, a small incision is formed at the site where the internal organ or tissue is to be operated because of inserting and guiding a tubular catheter (such as a cannula and trocar), and one or more surgical instruments are introduced through a single tubular catheter to perform the surgery. Since only a few fine holes are formed for inserting the working instruments, minimally invasive surgery causes less injury to the patient, faster healing period and less damage.

Further, medical endoscopes and accessories thereof can enter the human body through various tubes for observation or as surgical equipment. It can be widely used in the human body's throat, stomach, intestines, gallbladder and other digestive systems and urinary systems. Further, the conventional endoscope inspection is performed through a lens installed at one end of a flexible tube or through a rigid tube. Different endoscopes are matched with different types of endoscope lenses; the tube of the endoscope enters the body, so that the light source enters in front of the tube and an observer can observe through the lens. Then the data from the endoscope is transmitted to the observer and related electronic equipment to help the doctor's diagnosis and treatment. In addition, common accessories, such as clamps, surgeons can use different clamps to grasp, peel, cut or repair internal organs or internal tissues according to surgical needs. However, the commonly used endoscopes do not have the function of injecting medicines; surgeons need to go through other steps to inject medicines. The operation steps are cumbersome and increase the operation time; therefore, it is extremely easy to produce surgical risks.

In view of this, the inventor has invested a lot of research, development and effort, making breakthroughs and innovations, hoping to solve the current shortcomings with novel technical methods, not only bringing better products to the society, but also promoting industrial development at the same time.

### SUMMARY OF THE INVENTION

In view of the above needs, the main purpose of the present invention is to provide an endoscope kit having functions of injection, clamping, and placing medical materials or medicines. When the endoscope is positioned, it can be injected directly, and the internal organs or internal tissues can be grasped, peeled, cut or repaired. Furthermore, the wound produced is small, and no additional treatment is required after the operation thus shortening the recovery period.

To achieve the above objective, the present invention provides an endoscope kit having functions of injection, clamping, and placing medical materials or medicines, and the endoscope kit comprises a working instrument, an endoscope and Y joint; wherein the working instrument comprises an operation part, a rod portion and a handle, and the operation part and the handle are respectively connected to the two ends of the rod portion. Secondly, the endoscope comprises a cannula, a first light source input end, a first output and an injection needle; wherein the cannula has a lens and a tube part, the lens is installed at one front end of the cannula, and the lens is used to capture images. The first light source input end is used to input a light source and transmit the light source to the tube part; the tube part is located in the injection needle; and the front end of the tube part has a first surface. Furthermore, the Y joint is used to connect the cannula and the injection needle to the first light source input end and the first output; wherein the operation part of the working instrument enters the injection needle by means of the first output or the first light source input end through the Y joint.

In some embodiments, the injection needle further includes an optical component which is installed in front of the lens.

In some embodiments, the optical component is a hollow round tip triangular structure composed of a parallel plate and a semi-cylinder with an inclined surface.

In some embodiments, the optical component is a solid round tip triangle structure, and a slope part of the solid round tip triangular structure has a stepped processing plane. Further, the stepped processing plane is partially or completely disposed on the slope part, and the stepped processing plane can be a micron-level structure or a nano-level structure.

In some embodiments, the first surface of the injection needle is a flat surface, an inclined surface, a cone surface, or a circular arc surface.

In some embodiments, the Y joint has a first branch, a second branch, and a third branch; wherein the first branch has two interfaces for connecting the cannula and the injection needle to the first output.

In some embodiments, one end of the second branch is connected to the first branch; and another end of the second branch has an injection interface for injecting a medicine into the injection needle. Further, one end of the third branch is connected to the first branch, and another end of the third branch is connected to the first light source input end.

In some embodiments, the endoscope comprises a switching mechanism connected to a second output, and the switching mechanism is an optical fiber light collection structure, an image transmission switching mechanism or a power source required for an LED light source; and the switching mechanism includes a wireless transmission module or traditional wire transmission.

In some embodiments, the working instrument is a biopsy forceps, an electrocoagulation instrument, an ultrasonic scalpel, an electric cautery or a laser optical fiber instrument or a combination thereof.

In some embodiments, when the working instrument is the biopsy forceps, the operation part of the biopsy forceps has at least two clamping parts and a fixing part, the two clamping parts are connected with the fixing part, and the fixing part is connected with the rod portion.

In some embodiments, the output is connected to an output device, and the output device includes a communication device, a control device, and a display device. The communication device is coupled to the lens and is used for sending the images captured by the lens. Further, the control device is communicatively connected to the communication device to receive the images sent by the communication device, and send the images to the display device after processing. Furthermore, the display device is coupled to the control device to receive and show the images after processing; wherein the output is connected to the output device wirelessly, and the wireless connection includes Bluetooth, ZigBee, Wi-Fi or RF etc.

### BRIEF DESCRIPTION OF THE DRAWINGS

The above and other objectives, features and other advantages of the present invention will be more clearly understood from the following detailed description taken in conjunction with the accompanying drawings, in which:

FIG. 1 is an exploded view of a structure of an endoscope kit according to Embodiment 1 of the present invention;

FIG. 2A is an enlarged side perspective view of A in FIG. 1 according to the present invention;

FIG. 2B is one enlarged side perspective view of A in FIG. 1 according to the present invention;

FIG. 2C is another enlarged side perspective view of A in FIG. 1 according to the present invention;

FIG. 2D is the other enlarged side perspective view of A in FIG. 1 according to the present invention;

FIG. 3 is a schematic view of a structure of a working instrument according to the present invention;

FIG. 4A to 4H are schematic views of different operation parts according to the present invention;

FIG. 5 is an exploded view of a structure of an endoscope kit according to Embodiment 2 of the present invention;

FIG. 6 is one partial cross-sectional enlarged view of the injection needle according to the present invention;

FIG. 7 is another partial cross-sectional enlarged view of the injection needle according to the present invention; and

FIG. 8 is a schematic view of image output of endoscope according to the present invention.

### DETAILED DESCRIPTION OF THE PREFERRED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawings.

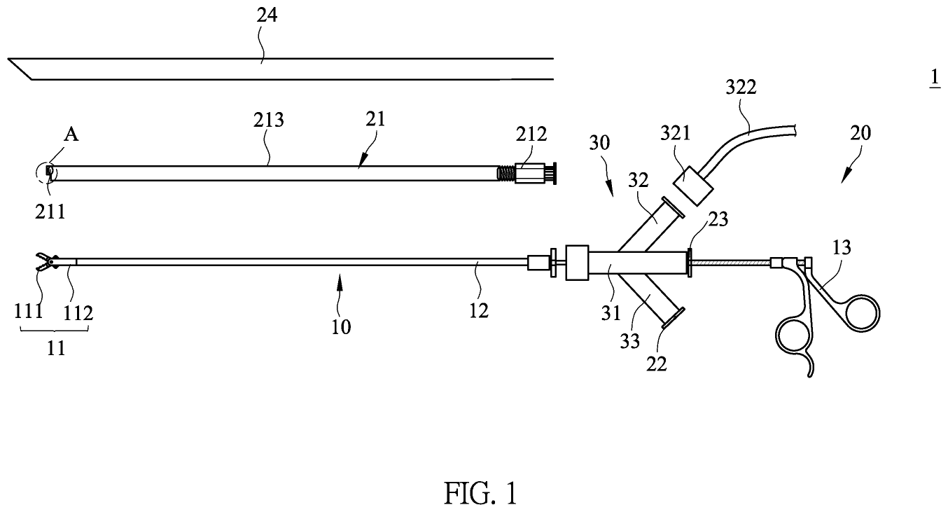

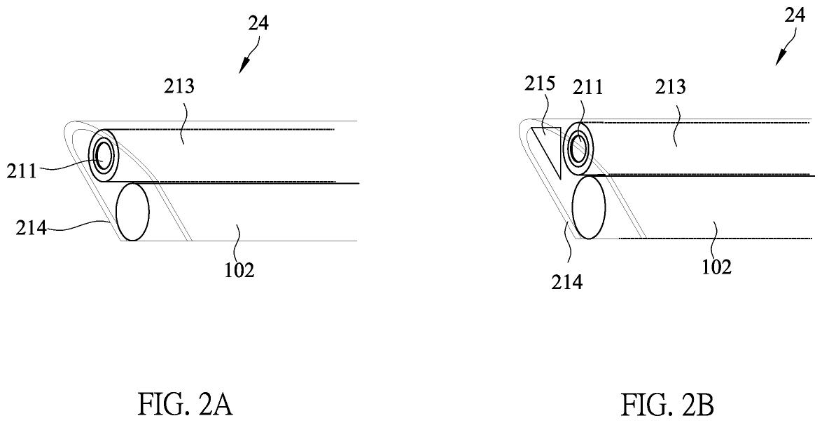

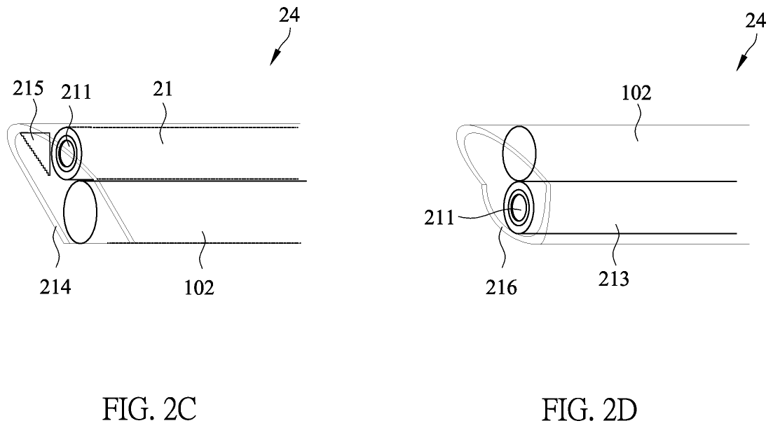

Please refer to FIG. 1 to FIG. 3, FIG. 1 is an exploded view of a structure of an endoscope kit according to Embodiment 1 of the present invention; FIG. 2A is an enlarged side perspective view of A in FIG. 1 according to the present invention; FIG. 2B is one enlarged side perspective view of A in FIG. 1 according to the present invention; FIG. 2C is another enlarged side perspective view of A in FIG. 1 according to the present invention; FIG. 2D is the other enlarged side perspective view of A in FIG. 1 according to the present invention; and FIG. 3 is a schematic view of a structure of a working instrument according to the present invention.

As shown in FIG. 1 to FIG. 3, the present invention provides an endoscope kit 1 having functions of injection, clamping, and placing medical materials or medicines, and the endoscope kit 1 comprises a working instrument 10, an endoscope 20 and Y joint 30; wherein the working instrument 10 comprises an operation part 11, a rod portion 12 and a handle 13, and the operation part 11 and the handle 13 are respectively connected to the two ends of the rod portion 12. Further, the style of the working instrument 10 can be randomly varied based on the user's requirements and preferences, and the working instrument 10 can be a biopsy forceps, an electrocoagulation instrument, an ultrasonic scalpel, an electric cautery or a laser optical fiber instrument or a combination thereof, but the invention is not limited thereto. In the present Embodiment, the working instrument 10 is the biopsy forceps; wherein the operation part 11 has at least two working instrumenting parts 111 and a connecting part 112, the at least two working instrumenting parts 111 are connected to the connecting part 112, the connecting part 112 is connected with the rod portion 12, and the at least two working instrumenting parts 111 are used to grasp biological tissue.

As shown in FIG. 1 to FIG. 2A, the endoscope 20 comprises a cannula 21, a first light source input end 22, a first output 23 and an injection needle 24;

wherein the cannula 21 can be movable or fixed inside the injection needle 10. Further, the cannula 21 has a lens 211, a head part 212 and a tube part 213, the lens 211 is installed at one front end of the cannula 21, and the lens 211 is used to capture images. Further, the lens can be a traditional optical image transmitter (for example, optical fiber, but the present invention is not limited thereto), a CCD (Charge Coupled Device) lens or a CMOS (Complementary Metal-Oxide Semiconductor) lens. Moreover, the tube part 213 is located in the injection needle 24; the front end of injection needle 24 has a first surface 214, the style of the first surface 214 may be randomly varied based on the user's requirements. In the present Embodiment, the first surface 214 is an inclined surface, but the present invention is not limited thereto. Furthermore, the first light source input end 22 is used to input a light source 101 and transmit the light source 101 to the tub part 213; and the light source can be an optical fiber beam or an LED light source. When the light source is an LED light source, the light source is located at the front end of the tube part 213, and an LED power cord is arranged in the tube part 213. In the present Embodiment, the light source 101 is the optical fiber beam, and the first light source input end 22 is used to input the optical fiber beam and transmit the optical fiber beam to the cannula 21. Furthermore, the diameter of the cannula 21 may be between 0.8 mm to 1.6 mm; and the diameter of the injection needle 24 may be between 1.2 mm to 2.5 mm.

Moreover, the Y joint 30 is used to connect the cannula 21 and the injection needle 24 to the first light source input end 22 and the first output 23; wherein the operation part 11 of the working instrument 10 enters the injection needle 24 by means of the first output 23 and the first branch 31 through the Y joint 30. Further, the Y joint 30 comprises has a first branch 31, a second branch 32, and a third branch 33; wherein the first branch 31 has two interfaces for connecting the injection needle 24 to the first output 23. The head part 212 of the cannula 21 is located inside the first branch 31; one end of the second branch 32 is connected to the first branch 31; and another end of the second branch 32 has an injection interface 321 for injecting a medicine 322. After medicine 321 is injected from the injection interface 321, the medicine 322 enters the first branch 31 through the second branch 32, and then flows into the injection needle 24, so that the medicine 322 enters the injection needle 24 through the first branch 31. Moreover, one end of the third branch 33 is connected to the first branch 31, and another end of the third branch 33 is connected to the first light source input end 22, so that the optical fiber beam 101 transmits to the cannula 21 through the first light source input end 22. In addition, the injection needle 24 further comprises a solid part 102, when the injection needle 24 enters the body, the solid part 102 is movably arranged in the injection needle 10 first, and the solid part 102 can prevent the body tissue from entering the injection needle 24. After the injection needle 24 enters the body, the solid part 102 is taken out, and the medicine is injected so that the medicine can be distributed in the injection needle 24.

As shown in FIG. 2B to FIG. 2D, the structure shown in FIG. 2B is substantially the same as the above FIG. 2A, but the differences are as follows. In FIG. 2B, the injection needle 24 further includes an optical component 215 which is installed in front of the lens 211; wherein the optical component 25 is a hollow round tip triangular structure composed of a parallel plate and a semi-cylinder with an inclined surface. The optical member 215 can be a glass or an optical plastic, and the optical member 215 can avoid the problem of optical refraction of the image captured by the lens 211. As shown in FIG. 2C, the structure shown in FIG. 2C is substantially the same as the above FIG. 2B, and the difference is that the optical component 25 of FIG. 2C is a solid round tip triangle structure, and a slope part of the solid round tip triangular structure has a stepped processing plane. Further, the stepped processing plane is partially or completely disposed on the slope part, and the stepped processing plane can be a micron-level structure or a nano-level structure. As shown in FIG. 2D, the structure shown in FIG. 2D is substantially the same as the above FIG. 2A, but the difference is the position of the lens 211; wherein the lens 211 of FIG. 2A is installed above the solid part 102, but the lens 211 of FIG. 2D is installed under the solid part 102. Furthermore, the front end of the injection needle 24 is a curved surface 216, such as a circular arc surface or a conical surface, at the front end of the injection needle 24 to avoid the problem of optical refraction. The lens 211 can be fixed in the cannula 21, or the lens 211 can be movably arranged in the cannula 21. Further, the lens 211 can be provided with a chip surrounding the optical fiber beam 101, and the chip can be used to transmit images to the first output 23.

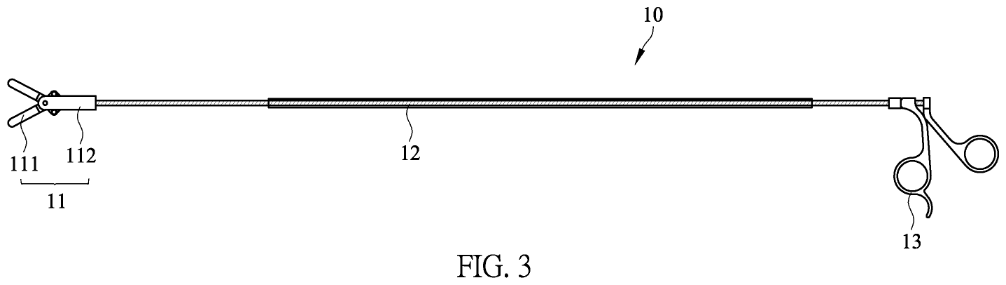

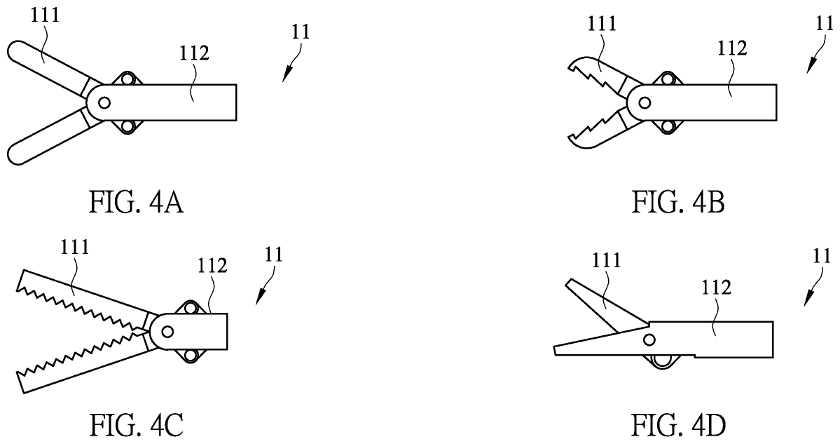

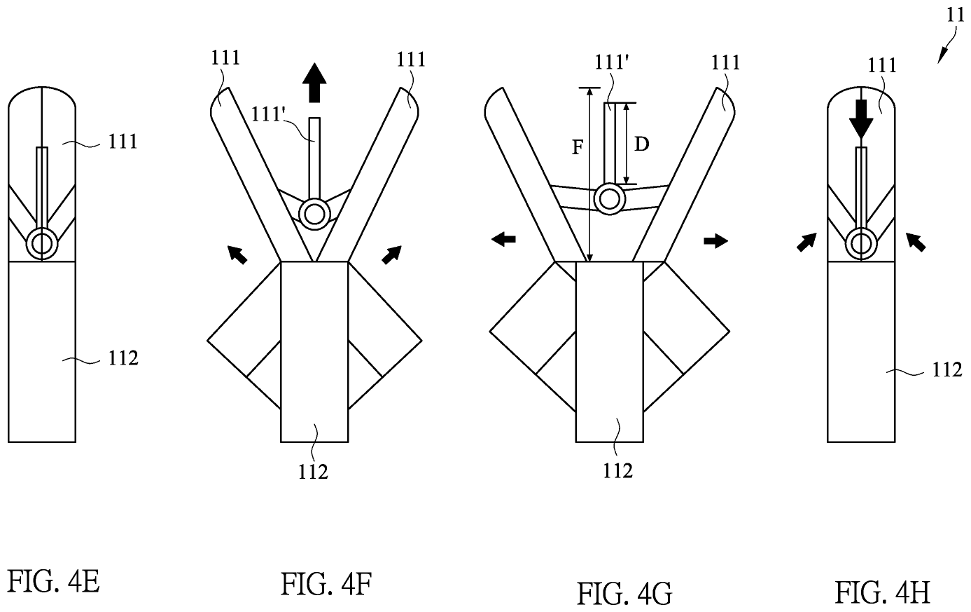

Please refer to FIG. 4A to FIG. 4H, FIG. 4A to 4H are schematic views of different operation parts according to the present invention.

As shown in FIG. 4A to FIG. 4H, the operation parts 11 have different styles as shown in FIG. 4A to FIG. 4H. For example, the operation parts 11 in FIG. 4A can be used for biopsy; the operation parts 11 in FIG. 4B can be used as saw teeth; the operation parts 11 in FIG. 4C can be used to grasp foreign bodies; the operation parts 11 in FIG. 4D can be used as scissors. The operation parts 11 of FIG. 4E to FIG. 4H is the same operation parts 11; wherein FIG. 4E shows the state in which the operation part 11 is not opened. FIG. 4F shows the state in which the operation part 11 is half-opened, the at least two working instrumenting parts 111 can open the biological tissue apart and then extend a solid part 111′ into the biological tissue, and the arrows in FIG. 4F represent the at least two working instrumenting parts 111 and the moving direction of the solid part 111′. Further, FIG. 4G shows the state in which the at least two working instrumenting portions 111 and the solid part 111′ are fully opened; and FIG. 4H shows the closed state of the at least two working instrumenting parts 111 and the solid part 111′. Furthermore, the styles of the operation parts 11 may be randomly varied based on the user's requirements, but the present invention is not limited thereto. All working instruments that can be applied to human internal organs or internal tissues for grasping, peeling, cutting or repairing are covered in the present application. Secondly, the shape of the top end of the solid part 111′ can be randomly varied based on the user's requirements and preferences, and the top shape of the solid part 111′ can be rectangular, shield-shaped, pointed or saw tooth shaped, but the invention is not limited thereto. Furthermore, as shown in FIG. 4G the length of an internal space of the at least two working instrumenting parts 111 is F, and the length of the solid part 111′ is D. When 0.5 F<D<F, the operation parts 11 is suitable for piercing harder tissues to take out or pushing medical materials or medicines; when D is less than 0.5 F, the operation parts 11 is suitable for placing larger medicines in the internal space; and when D is greater than or equal to 0.5 F, the operation parts 11 is suitable for placing smaller medicines in the internal space.

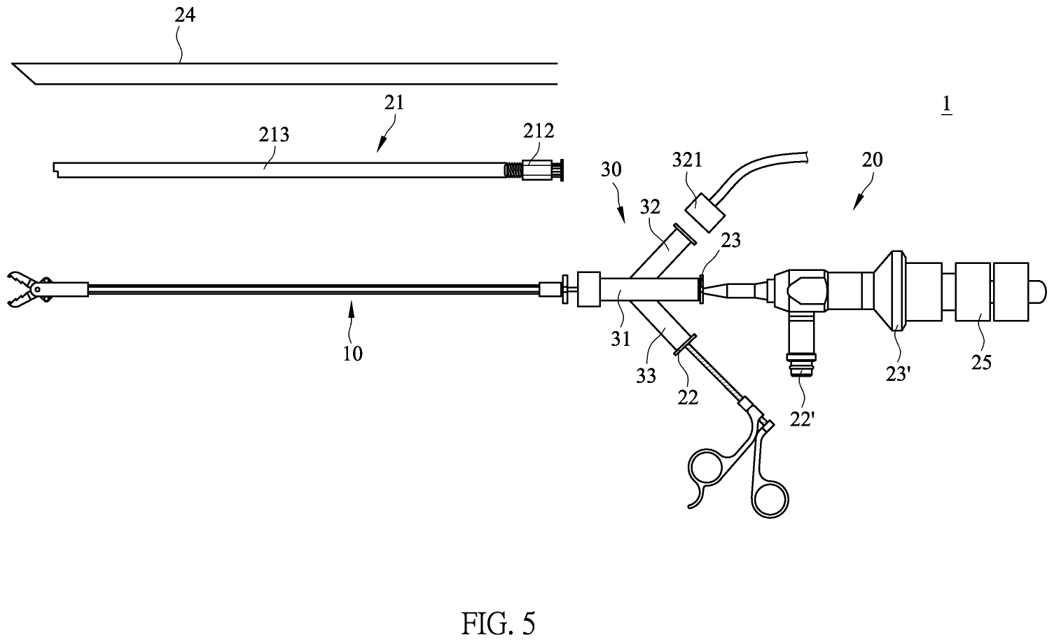

Please refer to FIG. 5, FIG. 5 is an exploded view of a structure of an endoscope with injection and working instrumenting function according to Embodiment 2 of the present invention.

As shown in FIG. 5, the second embodiment of the present invention is substantially the same as the endoscope described in the first embodiment. The difference is that the endoscope 20 further comprises a switching mechanism 25, a second light source input end 22′ and a second output 23′. Further, one end of the second light source input end 22′ is connected to the first output 23; another end is connected to the second output 23′; and the switching mechanism 25 is connected to the second output 23′. In the present Embodiment, the operation parts 11 of the working instrument 10 enters the injection needle 24 through the first light source input end 22, the third branch 33 and the first branch 31. The rod portion 12 is a soft member, so that the operation parts 11 and the rod portion 12 can enter the injection needle 24 through the Y joint 30. Furthermore, the second light source input end 22′ is used to input the light source and send the light source to the tube part 213. The switching mechanism 25 is an optical fiber light collection structure, an image transmission switching mechanism or a power source required for an LED light source; and the switching mechanism 25 includes a wireless transmission module or traditional wire transmission.

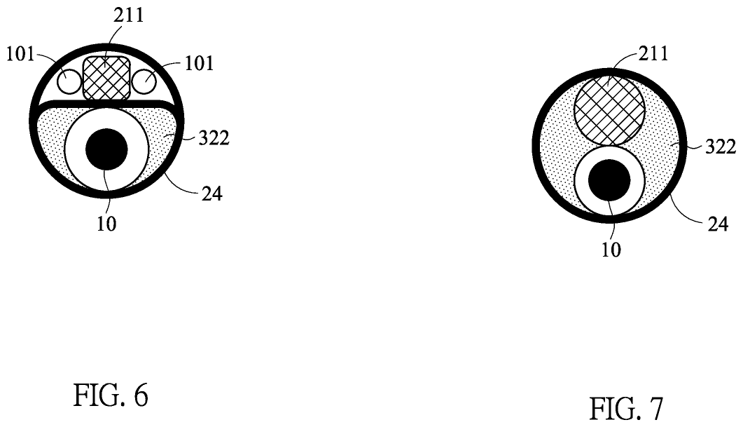

Please refer to FIG. 6, FIG. 6 is a partial cross-sectional enlarged view of the injection needle according to the present invention.

As shown in FIG. 6, the optical fiber beam 101 is located on two sides of the lens 211. When the medicine 322 is injected from the injection port 321, the medicine 322 is distributed on the periphery of the injection needle 24, and the working instrument 10 is arranged in the injection needle 24.

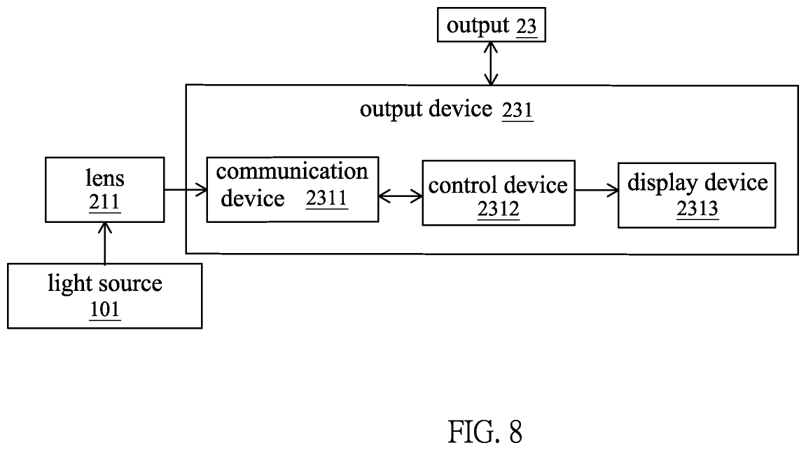

Please refer to FIG. 7, FIG. 7 is a partial cross-sectional enlarged view of the injection needle according to the present invention.

As shown in FIG. 7, the working instrument 10 is arranged in the injection needle 24, the lens 211 is located above the working instrument 10, and the medicine 322 is distributed around the injection needle 24 and the periphery of the lens 211; wherein the diameter of lens can be 0.6 mm and the length is 1 m to 2 m.

Please refer to FIG. 8, FIG. 8 is a schematic view of image output of endoscope according to the present invention.

As shown in FIG. 8, the first output 23 can be an eyepiece, or the first output 23 can be connected to an output device 231; wherein the output device 231 includes a communication device 2311, a control device 2312, and a display device 2313, and the communication device 2311 is coupled to the lens 211 for sending the images captured by the lens 211. Further, the control device 2312 is communicatively connected to the communication device 2311 to obtain the images sent by the communication device 2311 and send the images to the control device 2312 for processing. Moreover, the display device 2313 is coupled to the control device 233 for sending the processed images to the display device 2313; therefore, the doctor can observe the body through the lens 211, and send the data back to the communication device 2311 to further perform image processing and display the results on the display device 2313 to facilitate doctors' diagnosis and treatment. In one embodiment, the first output 23 can be connected to the output device 231 wirelessly, and the wireless connection can include Bluetooth, ZigBee, or Wi-Fi, but the invention is not limited thereto. Secondly, the output device 231 can be a display screen, a portable electronic device or a VR glasses. The doctor can watch the image captured by the lens by wearing the VR glasses, but the creation is not limited thereto.

The foregoing description, for the purpose of explanation, has been described with reference to specific embodiments. However, the embodiments were chosen and described in order to best explain the principles of the disclosure and its practical applications, to thereby enable others skilled in the art to best utilize the disclosure and various embodiments with various modifications as are suited to the particular use contemplated. The embodiments depicted above and the appended drawings are exemplary and are not intended to be exhaustive or to limit the scope of the disclosure to the precise forms disclosed. Modifications and variations are possible in view of the above teachings.

I claim:

1. An endoscope kit having functions of injection, clamping, and placing medical materials or medicines, comprising:
a working instrument comprising an operation part, a rod portion and a handle, and the operation part and the handle respectively connected to the two ends of the rod portion;
an endoscope comprising:
a cannula having a lens and a tube part, the lens installed at one front end of the cannula and the lens used to capture images;
a first light source input end used to input a light source and transmit the light source to the cannula;
a first output; and
an injection needle, the cannula located inside the injection needle, and the front end of the injection needle having a first surface; and
Y joint used to connect the cannula and injection needle to the first light source input end and the first output;
wherein the operation part of the working instrument enters the injection needle by means of the first output or the first light source input end through the Y joint.

2. The endoscope kit mentioned in claim 1, wherein the injection needle further includes an optical component which is installed at front of the lens.

3. The endoscope kit mentioned in claim 2, wherein the optical component is a hollow round tip triangular structure composed of a parallel plate and a semi-cylinder with an inclined surface.

4. The endoscope kit mentioned in claim 2, wherein the optical component is a solid round tip triangle structure, a slope part of the solid round tip triangular structure has a stepped processing plane, the stepped processing plane is partially or completely disposed on the slope part, and the stepped processing plane can be a micron-level structure or a nano-level structure.

5. The endoscope kit mentioned in claim 1, wherein the first surface is a flat surface, an inclined surface, a conical surface, or a circular arc surface.

6. The endoscope kit mentioned in claim 1, wherein the Y joint has a first branch, a second branch and a third branch; the first branch has two interfaces for connecting the cannula and the injection needle to the first output; one end of the second branch is connected to the first branch; and another end of the second branch has an injection interface for injecting a medicine into the injection needle; one end of the third branch is connected to the first branch, and another end of the third branch is connected to the first light source input end.

7. The endoscope kit mentioned in claim 1, wherein the first output is connected to an output device, the output device includes a communication device, a control device, and a display device; the communication device is coupled to the lens and is used for sending the images captured by the lens; wherein the control device is communicatively connected to the communication device to receive the images sent by the communication device, and send the images to the display device after processing; and the display device is coupled to the control device to receive and show the images after processing.

8. The endoscope kit mentioned in claim 1, wherein the endoscope comprises a switching mechanism connected to a second output; the switching mechanism is an optical fiber light collection structure, an image transmission switching mechanism or a power source required for an LED light source; and the switching mechanism includes a wireless transmission module or traditional wire transmission.

9. The endoscope kit mentioned in claim 1, wherein the working instrument is a biopsy forceps, an electrocoagulation instrument, an ultrasonic scalpel, an electric cautery or a laser optical fiber instrument or a combination thereof.

10. The endoscope kit mentioned in claim 9, wherein when the working instrument is the biopsy forceps, the operation part of the biopsy forceps has at least two clamping parts and a fixing part, the at least two clamping parts are connected to the fixing part, and the fixing part is connected with the rod portion.
